(19) United States
(12) Patent Application Publication
(10) Pub. No.: US 2020/0078563 A1
(43) Pub. Date: Mar. 12, 2020

(54) INTRAVENOUS (IV) ATTACHMENT THAT REDUCES ANXIETY AND/OR FEAR

(71) Applicants: Melissa Jones, Dickinson, TX (US); Garbrielle Nichols, Dickinson, TX (US); Heather Pond, La Porte, TX (US)

(72) Inventors: Melissa Jones, Dickinson, TX (US); Garbrielle Nichols, Dickinson, TX (US); Heather Pond, La Porte, TX (US)

(21) Appl. No.: 16/562,922

(22) Filed: Sep. 6, 2019

Related U.S. Application Data
(60) Provisional application No. 62/730,439, filed on Sep. 12, 2018.

(51) Int. Cl. A61M 25/02; A61M 25/06

(52) U.S. Cl. CPC A61M 25/02; A61M 2025/0246; A61M 25/0606

(57) ABSTRACT

The present invention includes a device and a method for reducing the anxiety and/or fear of a patient to an intravenous (IV) attachment, the IV attachment comprising: a figurine that a child, or a person with fear and/or anxiety find as cute, whimsical, appealing, or that reduces their fear and/or anxiety; and an IV ending that connects and closes an end of an IV assembly.

### CROSS-REFERENCE TO RELATED APPLICATIONS

This application claims priority to U.S. Provisional Application Ser. No. 62/730,439, filed Sep. 12, 2018, the entire contents of which are incorporated herein by reference.

### TECHNICAL FIELD OF THE INVENTION

The present invention relates in general to the field of intravenous (IV) administration of fluids, and more particularly, to a device that decreases the intimidation of IV lines to pediatric and other subjects with a fear of IVs.

### STATEMENT OF FEDERALLY FUNDED RESEARCH

None

### BACKGROUND OF THE INVENTION

Without limiting the scope of the invention, its background is described in connection with intravenous administration of fluids.

One such patent is U.S. Pat. No. 5,131,412, issued to Ranking, which is entitled “Pediatric Intravenous Device”. This inventor is said to teach an improved pediatric device for administering intravenous medications and fluids that maintains an infant or toddler's hand, arm, leg or foot in a stationary position to prevent the child from inadvertently dislodging the needle from the vein during sudden movement. The device is said to be anatomically correct for the pediatric patient's age and weight and includes a visco-elastic support molded to conform to the natural contours of the child, thus, keeping the extremity immobile.

Another such patent is U.S. Pat. No. 7,077,142, issued to Barany and Miller, which is entitled “Intravenous Retainment Immobilizer for Children”. These inventors are said to teach an intravenous catheter retainment device for use on young children and babies that supports and overlies a portion of the forearm and hand into which an intravenous needle is inserted. An overlying portion of a cover is a stretchable and see-through netting that conforms to the hand and lower forearm securely holding it in place on a rigid support board.

However, a need remains for devices that reduce the fear and anxiety caused by intravenous lines. The above devices restrain the subject, thereby increasing the anxiety associated with intravenous administration of liquids. What is needed is a device that does not restrain the patient, but that also reduced the anxiety and fear associated with intravenous lines, whether temporary or long-term.

### SUMMARY OF THE INVENTION

In one embodiment, the present invention includes an intravenous (IV) attachment for catheters that reduces anxiety and/or fear comprising, consisting essentially of, or consisting of: a figurine that a child, or a person with fear and/or anxiety find as cute, whimsical, appealing, or that reduces their fear and/or anxiety; and an IV ending that connects and closes an end of an IV assembly. In one aspect, the figurine is selected from animals, animal heads, action figures, whimsical figures, fantastical figures, whacky figures, colorful figures, arbitrary shapes, movie, play, book, cartoon or other fictional or non-fictional characters. In another aspect, the figurine is an image, such as a photograph, of something that helps soothe the patient, distract the patient, or provide a sense of relief, or causes the patient to provide assurance, cheer, comfort, console, reassure, solace, or to comfort the patient in need of the IV that is inserted into a slot connected to the IV ending. In another aspect, the IV ending is a luer lock ending, that can be connected temporarily, semi-permanently, or permanently. In another aspect, the figurine produces sounds or noises when manipulated. In another aspect, the figurine comprises a resilient material that can be squeezed by the patient. In another aspect, the figurine comprises at least one of: (1) a resilient rubber material, a rubberlike material, a flexible polymer, a synthetic polymer, silicone, or vinyl; (2) a solid, a partially solid, a gel-like, or a hollow interior or body; or (3) a solid core and an outer layer that is soft, plush, compliant, and/or resilient. In another aspect, the figurine comprises a material that is sterilizable by autoclaving, ozone, ionic or other radiation, or other method of sterilization. In another aspect, the figurine comprises a material that is polymers selected from ethylene vinyl acetate, derivatives of polypropylene, polyethylene, polyurethane, rayon, nylon, polyesters, polymethacrylate, silicon polymers, teflon, elastomers of polybutadiene/styrene, cis-polybutadiene, butyl rubber, ethylene-propylene rubber, polyisoprene, polyvinyl chloride (PVC), silicone, polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivativized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide-co-caprolactone) and/or derivatives thereof. In another aspect, the figurine is made by injection molding, compression molding, extrusion, and/or thermoforming techniques, and combinations thereof. In another aspect, the figurine is made from two or more parts joined using a permanent adhesive, thermal bonds, ultrasonic bonds, spot welds, thermal weld points, a stitch or stitches, strip welds, tacks formed by crimping, and/or press fit, shrink fit, sliding fit, interference fit, snap fit mechanisms, and combinations thereof. In another aspect, the figurine and the luer lock ending are formed concurrently, or are formed in two parts and joined with an permanent adhesive, thermal bonds, ultrasonic bonds, spot welds, thermal weld points, a stitch or stitches, strip welds, tacks formed by crimping, and/or press fit, shrink fit, sliding fit, interference fit, snap fit mechanisms, and combinations thereof. In another aspect, the figurine is designed not to be swallowed.

In one embodiment, the present invention includes a method of reducing the anxiety and/or fear of a patient to an intravenous (IV) device comprising, consisting essentially of, or consisting of: connecting permanently a figurine to an IV ending, wherein the figurine is selected to be cute, whimsical, appealing, or that reduces their fear and/or anxiety, wherein the IV ending can connect and close an end of an IV assembly during use of the IV. In one aspect, the figurine is selected from animals, animal heads, action figures, whimsical figures, fantastical figures, whacky figures, colorful figures, arbitrary shapes, movie, play, book, cartoon or other fictional or non-fictional characters. In another aspect, the figurine is an image, such as a photograph, of something that helps soothe the patient, distract the patient, or provide a sense of relief, or causes the patient to provide assurance, cheer, comfort, console, reassure, solace, or to comfort the patient in need of the IV that is inserted into a slot connected to the IV ending. In another aspect, the figurine comprises at least one of: (1) a resilient rubber material, a rubberlike material, a flexible polymer, a synthetic polymer, silicone, or vinyl; (2) a solid, a partially solid, a gel-like, or a hollow interior or body; or (3) a solid core and an outer layer that is soft, plush, compliant, and/or resilient. In another aspect, the figurine comprises a material that is sterilizable by autoclaving, ozone, ionic or other radiation, or other method of sterilization. In another aspect, the figurine comprises a material that is polymers selected from ethylene vinyl acetate, derivatives of polypropylene, polyethylene, polyurethane, rayon, nylon, polyesters, polymethacrylate, silicon polymers, teflon, elastomers of polybutadiene/styrene, cis-polybutadiene, butyl rubber, ethylene-propylene rubber, polyisoprene, polyvinyl chloride (PVC), silicone, polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivativized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide-co-caprolactone) and/or derivatives thereof. In another aspect, the figurine is made by injection molding, compression molding, extrusion, and/or thermoforming techniques, and combinations thereof.

### BRIEF DESCRIPTION OF THE DRAWINGS

For a more complete understanding of the features and advantages of the present invention, reference is now made to the detailed description of the invention along with the accompanying figures and in which:

FIG. 1 shows the entire assembly of an intravenous line, and the device of the present invention.

FIG. 2 shows one embodiment of the present invention, in which an elephant head attachment is shown as an example.

FIG. 3 shows a child with an IV and one embodiment of the present invention, in which an elephant head attachment is shown as an example.

FIGS. 4 and 5 show a close up view of the arm of a subject with the device of the present invention.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

Conventional IVs can be scary and intimidating to children. Pediatric patients are often anxious and afraid when they encounter an IV and often equate the IV to pain. The fear of the IV may lead some children to attempt to remove the IV line. It is important that an IV is locked and sterile, and can be used as needed. A suitable solution is desired.

The present invention provides an attachment for IV catheters that is in the shape of a figurine that a child, or a person with fear and/or anxiety with regard to IVs, will find as cute, whimsical, appealing, or that reduces their fear and/or anxiety. Figurines can include, for example, animals, animal heads, action figures, whimsical figures, fantastical figures, whacky figures, colorful figures, arbitrary shapes, movie, play, book, cartoon or other fictional or non-fictional characters. In another embodiment, the figurine could be in the form of a frame in which a parent or the patient inserts an image, such as a photograph, of something that helps soothe the patient, distract the patient, or provide a sense of relief, or causes the patient to provide assurance, cheer, comfort, console, reassure, solace, or to comfort the patient in need of the IV.

Each of these figurines is attached to a luer lock ending (or equivalent), that allows the figuring to be displayed to the patient to reduce anxiety and/or fear. The luer lock ending is used to close-off the IV, for example, when the IV is inserted temporarily, semi-permanently, or permanently. By providing the luer lock ending for standard biocompatible tubing, the device of the present invention serves two purposes, to maintain sterility of fluids in the IV line, while also reducing the anxiety and/or fear of the patient.

In certain embodiments, the figurine is configured to produce sounds or noises when manipulated. For example, in some embodiments, when the figurine is an animal, such as a dog or a cat, manipulation of the figurine could cause a dog or cat sound. The figurines can be made from a resilient material, which can be squeezed by the patient. In another embodiment, the figurine may be configured to produce sounds or noises when manipulated by the patient.

Typically, the figurine can be made from a resilient rubber material, a rubberlike material, a flexible polymer, a synthetic polymer, silicone, or vinyl. The device can have a solid, a partially solid, a gel-like, or a hollow interior or body. The device can have a solid core and an outer layer that is soft, plush, compliant, and/or resilient. The material is selected to be sterilizable, using any method for sterilization, such as autoclaving, ozone, ionic or other radiation, etc. In some embodiments, the elastic or flexible material (such as a natural or synthetic flexible material) is configured to recover its shape after a deformation (such as after a compression from a squeeze). Generally, the device of the present invention will be packaged for individual and/or single use. Polymers can include, e.g., ethylene vinyl acetate, derivatives of polypropylene, polyethylene, polyurethane, rayon, nylon, polyesters, polymethacrylate, silicon polymers, teflon, elastomers of polybutadiene/styrene, cis-polybutadiene, butyl rubber, ethylene-propylene rubber, polyisoprene, polyvinyl chloride (PVC), silicone, polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivativized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide-co-caprolactone) and/or derivatives thereof.

The figurines can be made by a suitable or known process or processes, such as injection molding, compression molding, extrusion, and/or thermoforming techniques, and combinations thereof. If made from two or more parts, the figurine can be joined using any suitable or known process or processes, such as, permanent adhesive, thermal bonds, ultrasonic bonds, spot welds, thermal weld points, a stitch or stitches, strip welds, tacks formed by crimping, and/or press fit, shrink fit, sliding fit, interference fit, and/or snap fit mechanisms, and so forth, and combinations thereof. Likewise, the figurine and the luer lock ending can be formed concurrently, or formed in two parts and joined using any suitable or known process or processes, such as, permanent adhesive, thermal bonds, ultrasonic bonds, spot welds, thermal weld points, a stitch or stitches, strip welds, tacks formed by crimping, and/or press fit, shrink fit, sliding fit, interference fit, and/or snap fit mechanisms, and so forth, and combinations thereof. Again, patient safety is a priority for the device of the present invention and any of the parts will be designed and combined such that the parts cannot separate and be swallowed.

Also, the figurine will be sufficiently large that it eliminates the possibility of the patient swallowing the device, and the luer lock ending will be permanently attached to the figurine, also to eliminate the possibility of the patient swallowing the device of the present invention.

In this example, an animal, specifically an elephant head, is used for pediatric patients. The device ensures an intravenous catheter is saline locked and maintains a sterile line so that fluids can be administered as needed. It allows children to feel more comfortable with intravenous catheters in a hospital setting. The device offers a cute and silly distraction for children to minimize their fear of the IV. The present invention helps to prevent children from removing the IV out of fear.

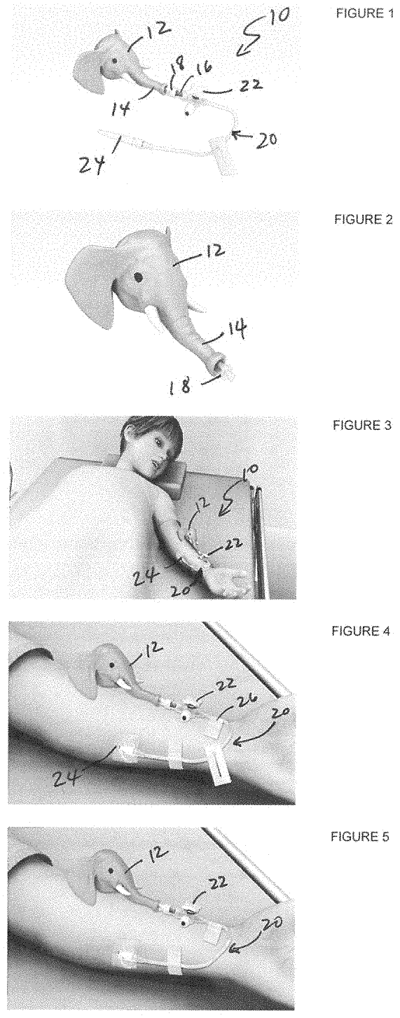

Referring now to FIGS. 1-5, the figurine IV 10 is a child-friendly attachment for an intravenous catheter. In this example of the invention, a gray elephant head 12 is made from a plastic. The gray elephant head 12 may be sideways facing and the trunk 14 will screw into one end of an approximately 3.5″ to 4″ plastic IV extension tube 16 via a luer lock ending 18 connected to an IV catheter assembly 20 that connects via a valve 22. The other end of the IV assembly 20 includes the needle 24, and can be affixed to a patient with biocompatible tape 26. Before use, the gray elephant head 12 can include a luer lock or screw-on cap to maintain sterility (not shown), and be individually packaged in a sterile environment. The cap can be removed for the tubing to fit into an IV catheter assembly 20. The gray elephant head 12 may come in a variety of other shapes/likeness if desired, as outlined hereinabove. The exact specifications may vary.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited features, elements, components, groups, integers, and/or steps, but do not exclude the presence of other unstated features, elements, components, groups, integers and/or steps. In embodiments of any of the compositions and methods provided herein, “comprising” may be replaced with “consisting essentially of” or “consisting of”. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only. As used herein, the phrase “consisting essentially of” requires the specified features, elements, components, groups, integers, and/or steps, but do not exclude the presence of other unstated features, elements, components, groups, integers and/or steps as well as those that do not materially affect the basic and novel characteristic(s) and/or function of the claimed invention.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

What is claimed is:

1. An intravenous (IV) attachment for catheters that reduces anxiety and/or fear comprising:
a figurine that a child, or a person with fear and/or anxiety find as cute, whimsical, appealing, or that reduces their fear and/or anxiety; and
an IV ending that connects and closes an end of an IV assembly.

2. The IV attachment of claim 1, wherein the figurine is selected from animals, animal heads, action figures, whimsical figures, fantastical figures, whacky figures, colorful figures, arbitrary shapes, movie, play, book, cartoon or other fictional or non-fictional characters.

3. The IV attachment of claim 1, wherein the figurine is an image, such as a photograph, of something that helps soothe the patient, distract the patient, or provide a sense of relief, or causes the patient to provide assurance, cheer, comfort, console, reassure, solace, or to comfort the patient in need of the IV that is inserted into a slot connected to the IV ending.

4. The IV attachment of claim 1, wherein the IV ending is a luer lock ending, that can be connected temporarily, semi-permanently, or permanently.

5. The IV attachment of claim 1, wherein the figurine produces sounds or noises when manipulated.

6. The IV attachment of claim 1, wherein the figurine comprises a resilient material that can be squeezed by the patient.

7. The IV attachment of claim 1, wherein the figurine comprises at least one of: (1) a resilient rubber material, a rubberlike material, a flexible polymer, a synthetic polymer, silicone, or vinyl; (2) a solid, a partially solid, a gel-like, or a hollow interior or body; or (3) a solid core and an outer layer that is soft, plush, compliant, and/or resilient.

8. The IV attachment of claim 1, wherein the figurine comprises a material that is sterilizable by autoclaving, ozone, ionic or other radiation, or other method of sterilization.

9. The IV attachment of claim 1, wherein the figurine comprises a material that is polymers selected from ethylene vinyl acetate, derivatives of polypropylene, polyethylene, polyurethane, rayon, nylon, polyesters, polymethacrylate, silicon polymers, teflon, elastomers of polybutadiene/styrene, cis-polybutadiene, butyl rubber, ethylene-propylene rubber, polyvinyl chloride (PVC), silicone, polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivativized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate cellulose acetate phthalate, carboxylethyl cellulose, polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide-co-caprolactone) and/or derivatives thereof.

10. The IV attachment of claim 1, wherein the figurine is made by injection molding, compression molding, extrusion, and/or thermoforming techniques, and combinations thereof.

11. The IV attachment of claim 1, wherein the figurine is made from two or more parts joined using a permanent adhesive, thermal bonds, ultrasonic bonds, spot welds, thermal weld points, a stitch or stitches, strip welds, tacks formed by crimping, and/or press fit, shrink fit, sliding fit, interference fit, snap fit mechanisms, and combinations thereof.

12. The IV attachment of claim 1, wherein the figurine and the luer lock ending are formed concurrently, or are formed in two parts and joined with an permanent adhesive, thermal bonds, ultrasonic bonds, spot welds, thermal weld points, a stitch or stitches, strip welds, tacks formed by crimping, and/or press fit, shrink fit, sliding fit, interference fit, snap fit mechanisms, and combinations thereof.

13. The IV attachment of claim 1, wherein the figurine is designed not to be swallowed.

14. A method of reducing the anxiety and/or fear of a patient to an intravenous (IV) device comprising:
connecting permanently a figurine to an IV ending, wherein the figurine is selected to be cute, whimsical, appealing, or that reduces their fear and/or anxiety, wherein the IV ending can connect and close an end of an IV assembly during use of the IV.

15. The method of claim 14, wherein the figurine is selected from animals, animal heads, action figures, whimsical figures, fantastical figures, whacky figures, colorful figures, arbitrary shapes, movie, play, book, cartoon or other fictional or non-fictional characters.

16. The method of claim 14, wherein the figurine is an image, such as a photograph, of something that helps soothe the patient, distract the patient, or provide a sense of relief, or causes the patient to provide assurance, cheer, comfort, console, reassure, solace, or to comfort the patient in need of the IV that is inserted into a slot connected to the IV ending.

17. The method of claim 14, wherein the figurine comprises at least one of: (1) a resilient rubber material, a rubberlike material, a flexible polymer, a synthetic polymer, silicone, or vinyl; (2) a solid, a partially solid, a gel-like, or a hollow interior or body; or (3) a solid core and an outer layer that is soft, plush, compliant, and/or resilient.

18. The method of claim 14, wherein the figurine comprises a material that is sterilizable by autoclaving, ozone, ionic or other radiation, or other method of sterilization.

19. The method of claim 14, wherein the figurine comprises a material that is polymers selected from ethylene vinyl acetate, derivatives of polypropylene, polyethylene, polyurethane, rayon, nylon, polyesters, polymethacrylate, silicon polymers, teflon, elastomers of polybutadiene/styrene, cis-polybutadiene, butyl rubber, ethylene-propylene rubber, polyvinyl chloride (PVC), silicone, polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivativized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate cellulose acetate phthalate, carboxylethyl cellulose, polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide-co-caprolactone) and/or derivatives thereof.

20. The method of claim 14, wherein the figurine is made by injection molding, compression molding, extrusion, and/or thermoforming techniques, and combinations thereof.
